(19) United States
(12) Patent Application Publication
(10) Pub. No.: US 2011/0238071 A1
(43) Pub. Date: Sep. 29, 2011

(54) DRILL ASSISTANCE KIT FOR IMPLANT HOLE IN A BONE STRUCTURE

(76) Inventor: Alain Fernandez-Scoma, Montastruc La Conseillere (FR)

(21) Appl. No.: 13/047,510

(22) Filed: Mar. 14, 2011

Related U.S. Application Data
(60) Provisional application No. 61/340,956, filed on Mar. 24, 2010.

(51) Int. Cl. A61B 17/56

(52) U.S. Cl. 606/80; 606/96

(57) ABSTRACT

The present invention provides a kit for bone surgery comprising a chirurgical drill and a base frame for positioning of said drill on an anatomical part of a patient. The base frame comprises at least one guiding tube with a cylindrical inner surface of predetermined diameter and also comprises a longitudinal cutout for direct view of the drilling site. The kit further comprises a burr-ring with a cylindrical outer surface of predetermined diameter arranged to be placed around the chirurgical drill. The diameter of the cylindrical outer surface of the burr-ring is slightly less than the diameter of the cylindrical inner surface of the guiding tube such that the burr-ring and the guiding tube are at most in a two-degree of freedom relationship along the longitudinal axis of the guiding tube when the burr-ring is engaged in the guiding tube. The invention also provides a bone surgery method using the kit.

### TECHNICAL FIELD

The present invention relates to a kit for bone surgery. More particularly, the present invention relates to a drill kit suitable for instance for the assistance of drilling a hole in a jaw bone structure for dental implants.

### BACKGROUND OF THE INVENTION

Drilling of chirurgical holes is known to be a delicate procedure that needs a high degree of precision. Each hole must be drilled precisely with regard on the depth and on the axial orientation of the chirurgical drill. This is particularly the case when it comes to chirurgical procedures that implicate a subsequent implant.

During chirurgical drilling procedures, drills often have to be exchanged. For example, some drilling procedures comprise several distinct steps in order to slowly enlarge the hole in which an implant is eventually placed. This implicates distinct drills of increasing diameters. Other drilling procedures use drills of different nature. Some procedures use specialized miniature percussion drills in combination with classic drills. Hence there is a need to allow easy exchange of the drills during the procedure.

Generally, a drilling procedure has an effect on the functionality of an implant. For instance, in the field of dentistry, chirurgical drilling is usually carried out in the lower and upper jaw that are both known to be hardly accessible. The drilling procedure thus is a crucial step to create a hole that can properly receive and carry a long-term implant.

For most chirurgical drilling procedures, it is required to ensure maximum preservation of the integrity of the bone structure during and after the drilling procedure. For this, it is advantageous to provide stability of the drilling means and to ensure that the operating person has a good visibility on the drilling site. Moreover, it is important to avoid overheating phenomena and to ensure a proper drill-dust evacuation.

Furthermore, drilling procedures are known to be delicate because they can lead to permanent lesions. In fact, there has to be taken great care not to interfere with the close nervous and/or muscular system.

The placement of one or more implants often is a solution of “last-resort” and consequently takes place after other attempts of therapeutic treatments have failed. This leads to the fact that the physiological drilling sites are often damaged which makes it even more difficult to engage a precise drilling procedure.

Also, in the case of chirurgical procedures in a buccal-region numerous other security measures are implicated in order to assure welfare of the patient. For example, there is a need to survey the risk of microbiological infections caused by surrounding bacteria.

U.S. Pat. No. 5,320,529 discloses a drill guide comprising drill guide bores. The drill guide is made from a stereolithographic model constructed from digital image data (computerized tomography) which allows the operating person to view the external and internal anatomy prior to the chirurgical drilling. However, many of the above cited drawbacks are not overcome by this drill guide, including visibility issues and/or dust evacuation for example.

WO 2008/149288 discloses a drill assistance device comprising a base with a guiding-bush in combination with a tubular guide sleeve. The tubular guide sleeve is arranged to fit specifically to a corresponding drill. The guide sleeve has to be inserted axially into a corresponding guiding-bush before the drilling procedure with a distinct drilling tool can begin. The whole system thus has to be adapted if one wants to exchange the chirurgical drill, since each chirurgical drill must fit into a corresponding guide sleeve.

None of the known prior art solutions is able to overcome properly the above cited drawbacks. Therefore, an improvement in chirurgical and mechanical drill-means is needed to allow a satisfying procedure that can overcome said drawbacks.

### SUMMARY OF THE INVENTION

The present invention overcomes the drawbacks given above and improves the general situation.

Particularly the present invention offers a great flexibility in drill exchange, overall visibility, broad access to the drilling site and sensitive control and regulation of the drilling procedure, including lateral positioning of the chirurgical drill and precise axial orientation of the latter during drilling.

For this, the invention provides a kit for bone surgery comprising a chirurgical drill and a base frame for positioning of said drill on an anatomical part of a patient, wherein said base frame comprises at least one guiding tube with a cylindrical inner surface having a predetermined diameter and a longitudinal cutout, the kit further comprising a burr-ring arranged to be placed around said chirurgical drill, said burr-ring comprising a cylindrical outer surface having a predetermined diameter, wherein the diameter of said cylindrical outer surface of the burr-ring is slightly less than said diameter of said cylindrical inner surface of the guiding tube and such that the burr-ring and the guiding tube are at most in a two-degree of freedom relationship along the longitudinal axis of the guiding tube when the burr-ring is engaged in the guiding tube, while the longitudinal cutout provides for direct view of the drilling site.

In a preferred embodiment the burr-ring comprises a central longitudinal hole of predetermined diameter substantially complementary to an outer diameter of said chirurgical drill so that said burr-ring can be placed around said chirurgical drill.

In another embodiment the burr-ring and said chirurgical drill are in a one-degree of freedom relationship when the burr-ring is placed around the chirurgical drill and wherein said one-degree of freedom relationship is a rotation around a common longitudinal axis.

In another embodiment the burr-ring and said chirurgical drill are mounted fixedly together in a zero-degree of freedom relationship when the burr-ring is placed around the chirurgical drill.

In another embodiment the burr-ring comprises clamping means comprising at least one rib arranged circumferentially of said central longitudinal hole adapted to engage in at least one slot arranged circumferentially on said chirurgical drill.

In another embodiment the burr-ring comprises an at least partially circumferential shoulder on an upper side to form an abutment together with the upper side of said guiding tube when the burr-ring is introduced in the guiding tube.

In another embodiment the two-degree of freedom relationship of said burr-ring and said guiding tube is set along a common longitudinal axis and consisting of a longitudinal translation and a rotation.

In another embodiment the burr-ring comprises at least one circumferential longitudinal rib on its cylindrical outer surface and the guiding tube comprises at least one circumferential longitudinal slot on its cylindrical inner surface so that the burr-ring and the guiding tube are in a one-degree of freedom relationship consisting of a longitudinal translation when said burr-ring is engaged in said guiding tube.

In another embodiment the burr-ring comprises at least one circumferential longitudinal slot on its cylindrical outer surface and the guiding tube comprises at least one circumferential longitudinal rib on its cylindrical inner surface so that the burr-ring and the guiding tube are in a one-degree of freedom relationship consisting of a longitudinal translation when said burr-ring is engaged in said guiding tube.

In a particular preferred embodiment the longitudinal cutout of the guiding tube extends along the entire guiding tube.

The present invention also provides a method for bone surgery wherein a kit according to one of the above embodiments is used. The method for bone surgery comprises the steps of:
a. placing a base frame comprising at least one guiding tube with a cylindrical inner surface having a predetermined diameter and a longitudinal cutout on an anatomical part of a patient for providing positioning of a chirurgical drill on said anatomical part,
b. placing a burr-ring around said chirurgical drill wherein said burr-ring comprises a cylindrical outer surface having a predetermined diameter that is slightly less than said diameter of said cylindrical inner surface of said guiding tube,
c. drilling into said anatomical part of the patient wherein the chirurgical drill penetrates axially into the anatomical part along a common central longitudinal axis while said burr-ring engages into said guiding tube and wherein the burr-ring and the guiding tube are at most in a two-degree of freedom relationship and wherein said two-degree of freedom relationship is consisting of a rotation and a translation of the burr-ring along said common central longitudinal axis, and
d. controlling the drilling by providing direct view of the drilling site by said longitudinal cutout of the guiding tube.

The invention also aims a burr-ring comprising a body of generally cylindrical shape having a central longitudinal hole of predetermined diameter wherein said central longitudinal hole comprises at least one rib consisting of a circumferential diameter constriction of predetermined length.

Preferentially, the body of the Burr-ring according to the invention has a cylindrical outer surface and comprises a shoulder that is at least partially arranged circumferential on said cylindrical outer surface. Optionally, the body comprises a terminal frustoconical part of a decreasing diameter and wherein said decreasing diameter decreases from substantially the diameter of said body to substantially the predetermined diameter of said hole.

The invention further provides a base frame comprising a foot structure homologous to an anatomical part of a patient and at least one guiding tube with a cylindrical inner surface of predetermined diameter, said guiding tube comprising a longitudinal cutout. The foot structure can be homologous to an anatomical part chosen in the group consisting of an upper jaw bone structure, a lower jaw bone structure or at least a part of a dentition

Preferentially, the longitudinal cutout of the base frame extends along the entire guiding tube.

Further features and advantages of the present invention will become apparent from the following detailed description and from the attached drawings.

### BRIEF DESCRIPTION OF THE DRAWINGS

FIG. 1 shows a perspective view of a burr-ring according to an embodiment of the present invention.

FIG. 2 shows a front view of the burr-ring of FIG. 1,

FIG. 3 shows a sectional view of the burr-ring of FIG. 1,

FIG. 4 shows a perspective view of a burr-ring according to another embodiment of the present invention,

FIG. 5 shows a front view of the burr-ring of FIG. 4,

FIG. 6 shows a sectional view of the burr-ring of FIG. 4,

FIG. 7 shows a perspective view of a burr-ring according to another embodiment of the present invention,

FIG. 8 shows a perspective view of a chirurgical drill according to an embodiment of the present invention,

FIG. 9 shows a front view of the chirurgical drill of FIG. 8,

FIG. 10 shows a perspective view of a chirurgical drill according to another embodiment of the present invention,

FIG. 11 shows a schematic perspective view of a non assembled kit according to an embodiment of the present invention,

FIG. 12 shows a schematic perspective view of an assembled kit according to an embodiment of the present invention before a drilling procedure,

FIG. 13 shows a schematic perspective view of an assembled kit according to an embodiment of the present invention during a drilling procedure,

FIG. 14 shows a picture of a perspective view a kit according to an embodiment of the present invention,

FIG. 15 shows a schematic picture of a perspective view of a base frame according to an embodiment of the present invention placed on a jaw bone structure,

FIG. 16 shows a picture of a top view of a base frame according to another embodiment of the present invention placed on a jaw bone structure and dentition,

FIG. 17 shows a picture of a perspective view of a burr-ring placed around a chirurgical drill combined with an electric motor tool, and

FIG. 18 shows a flowchart of a method for bone surgery according to the invention.

The attached drawings may not only provide a better understanding of the invention but in some cases may also contribute to its definition.

### DETAILED DESCRIPTION OF THE EXEMPLARY EMBODIMENTS

First it has to be noted that the present invention partially involves a foregoing tomographic method that comprises collecting of tomographic data that are representative of an anatomical zone. These data are mainly used to provide a foot structure (according to a homologous pattern such as a jaw bone structure for example) of the base frame of the present invention (see below for more details). This method is described in detail in the patent application U.S. Ser. No. 12/602,634 which is incorporated herein by reference.

Also, it has to be noted that the present invention is described with reference to a dentistry procedure. It has to be understood that the present invention is particularly adapted for this kind of procedure but can however be used in other fields of chirurgical procedures.

Further, it has to be noted that In the present description the wording “jaw bone”, “jaw bone structure”, “upper jaw bone” or “lower jaw bone” designate the entire maxilla, including the gum, and not only the bone structure of the jaw bone. The wording “dentition” designates the specific dentition of a patient that can comprise teeth or not.

The present invention usually requires a tomography of a patient on which a chirurgical procedure is to be made. The patient is subjected to a tomography in order to collect tomographic data that are representative of an anatomical part (or anatomical zone) of his body. The tomographic data provide information to build a three-dimensional solid reproduction of said anatomical part. The three-dimensional solid reproduction of the anatomical part is then used to build a rigid base frame, that is able to position and fix itself on said reproduction and consequently on said anatomical part of the patient's body. In order to allow proper positioning of the base frame on the anatomical part of the patient, the base frame comprises a foot structure that is substantially homologous to said anatomical part.

The three-dimensional solid reproduction of the anatomical part is preferably done by stereolithography in combination with tomography. Preferably, the reproduction is made of a translucent or transparent material so that one can visualize a penetration of a drill in a selected area. Preferably, the tomographic data may represent slices of said anatomical part with a distance less or equal to 500 μm and more preferably around 400 μm or less. Accordingly, subsequent procedure steps based on the tomographic data can be carried out with a precision level of equal or less than 500 μm.

The present invention mainly involves three distinct elements that are namely:
a base frame comprising and at least one guiding tube,
a burr-ring arranged to fit on a chirurgical drill, and
a chirurgical drill arranged to receive a burr-ring.

In the kit according to the invention, all above elements cooperate with one another.

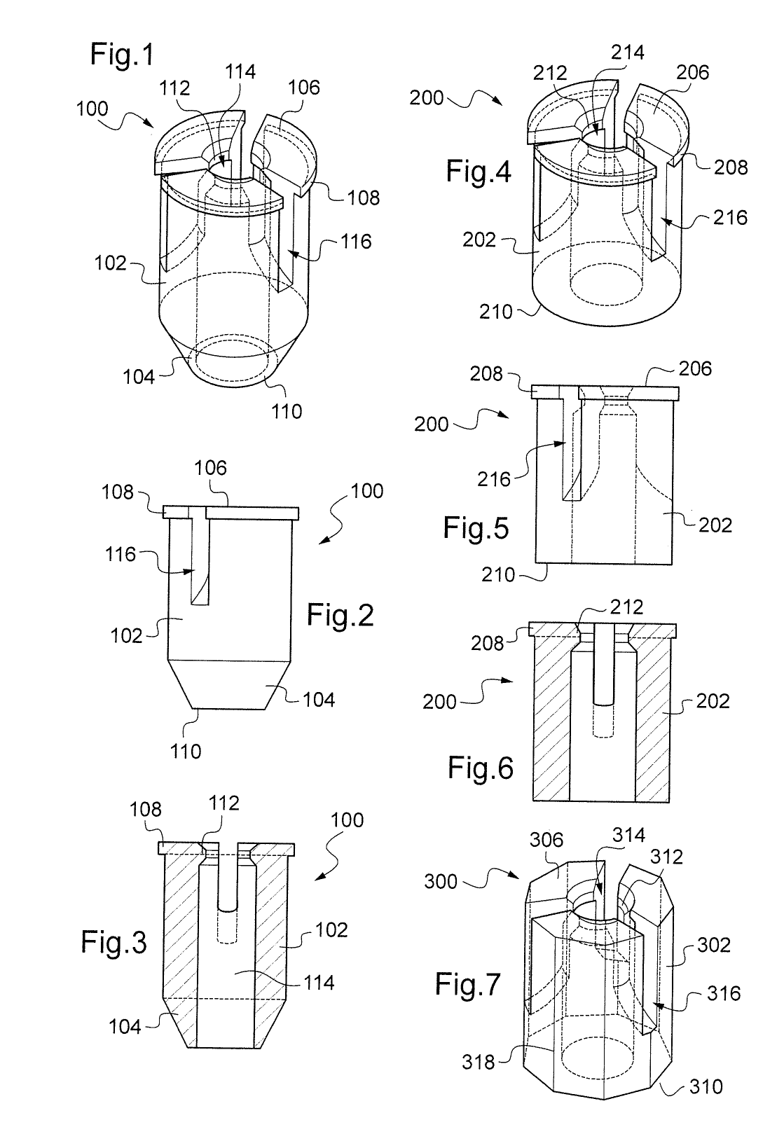

FIGS. 1-3 show a burr-ring according to an embodiment of the invention. The burr-ring 100 has an overall cylindrical shape and a total height comprised within a range going from 7 mm to 10 mm. In a preferred embodiment the total height of the burr-ring 100 is of 8 mm. More precisely, the burr-ring 100 comprises a cylindrical body 102 with a cylindrical outer surface of predetermined diameter. The predetermined diameter may be comprises within a range going from 4 mm to 6.5 mm. In a preferred embodiment the predetermined diameter of the cylindrical outer surface of the cylindrical body 102 is of 5 mm. The burr-ring 100 comprises an upper side 106 and a lower side 110 arranged at opposite terminal parts. Along its longitudinal axis, the burr-ring 100 comprises a central longitudinal hole 114 of predetermined diameter that can be within a range going from 2 mm to 3 mm. In a preferred embodiment the predetermined diameter of the central longitudinal hole 114 is of 2.40 mm.

Near its lower side 110, the burr-ring 100 of FIGS. 1-3 comprises a diameter restriction on its cylindrical outer surface of the body 102. The restriction defines a frustoconical part 104. The diameter restriction may be continuous going from 5-6.5 mm to 2.2-3 mm (depending of the predetermined diameters of the cylindrical outer surface and the central longitudinal hole respectively).

Near its upper side 106, the burr-ring 100 of FIGS. 1-3 comprises a circumferentially positioned shoulder 108 that is arranged to cooperate with a guiding tube 600 according to the invention as will become apparent below (FIGS. 11-13). The shoulder can be of variable dimensions and is preferentially about 0.5 mm to 4.5 mm height and extends about 0.5 mm to 1 mm over the predetermined diameter of the cylindrical outer surface.

The body 102 shown in FIGS. 1-3 comprises three cutaways gaps 116. In the shown embodiment these cutaways gaps are arranged to be engaged by a tripod gripper in order to dismantle the burr-ring 100 of a chirurgical drill.

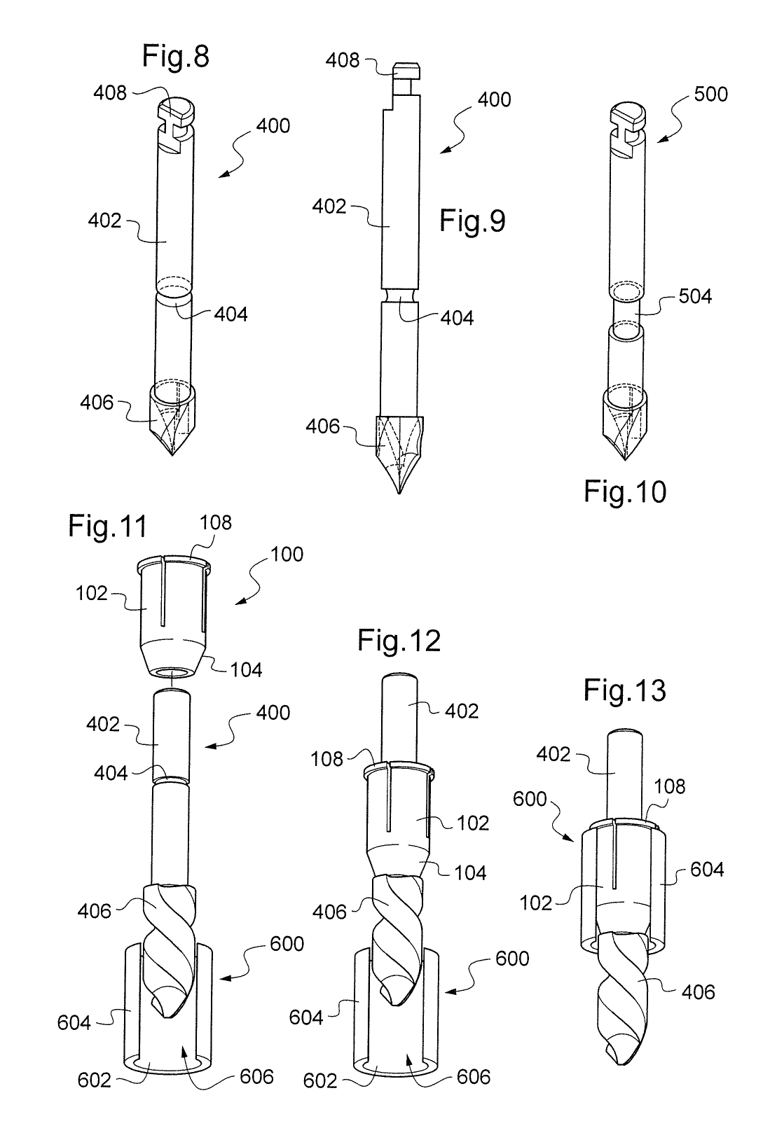

The cutaways gaps 116 also confer flexibility to the upper side of the burr-ring. The flexibility is dependent on the material used to build the burr-ring 100. Especially high flexibility is given when the burr-ring 100 is made of plastic material. In a preferred embodiment the plastic material is a thermoplastic material such as polyether ether ketone (PEEK). The flexibility is advantageous to mount easily the burr-ring 100 on a chirurgical drill 400 (see FIGS. 8-9) in combination with clamping means as described below; however, in other embodiments where the burr-ring 100 is mounted fixedly on a chirurgical drill, there is no need of any flexibility.

The burr-ring 100 comprises clamping means in order to be mounted on a chirurgical drill 400. The clamping means shown in FIGS. 1-3 comprise at least one rib 112 arranged circumferentially of said central longitudinal hole 114 adapted. Each rib 112 is arranged to engage in a corresponding slot 404 arranged circumferentially on a chirurgical drill 400 as shown in FIGS. 8, 9 and 11.

The ribs 112 are formed by a circumferential diameter constriction of the central longitudinal hole 114 wherein the constriction extends on a predetermined length and preferentially extends about 1 mm. The constriction may scale about 0.5 mm with reference to the predetermined diameter of the central longitudinal hole 114. In a preferred embodiment, the constriction is arranged so that the diameter of the central longitudinal hole 114 is constricted to 2.20 mm.

Accordingly, a homologous slot 404, in which the ribs 112 can engage, has to be arranged circumferentially on the chirurgical drill 400 as shown in FIGS. 8, 9 and 11. By engaging the slot 404, the ribs 112 clip onto the chirurgical drill 400 as it is shown in FIG. 12. Once the ribs 112 are clipped into the slot 404, the burr-ring 100 and the chirurgical drill 400 are clamped together and are at most in a one-degree of freedom relationship that is a rotation around a common central longitudinal axis.

In other embodiments as will become apparent hereinafter, the burr-ring 100 and the chirurgical drill 400 are rigidly fixed together and are in a zero-degree of freedom relationship. For this, the ribs and the slot are usually expanded in length in order to provide a greater connection surface between one another and enhance their adherence.

FIGS. 4-6 show a burr-ring 200 of another embodiment of the invention. The burr-ring 200 has an overall cylindrical shape and a total height comprised within a range going from 7 mm to 10 mm. In a preferred embodiment the total height of the burr-ring 200 is of 8 mm. The burr-ring 200 comprises a cylindrical body 202 with a cylindrical outer surface of predetermined diameter. The predetermined diameter may be comprises within a range going from 4 mm to 6.5 mm. In a preferred embodiment the predetermined diameter of the cylindrical outer surface of the cylindrical body 202 is of 5 mm. The burr-ring 200 comprises an upper side 206 and a lower side 210 arranged at opposite terminal parts. Along its longitudinal axis, the burr-ring 200 comprises a central longitudinal hole 214 of predetermined diameter that can be within a range going from 2 mm to 3 mm. In a preferred embodiment the predetermined diameter of the central longitudinal hole 214 is of 2.40 mm.

Contrary to what has been described for the burr-ring 100 of FIGS. 1-3, the burr-ring 200 of FIGS. 4-6 is missing a frustoconical part and consequently has an overall cylindrical shape. Near its upper side 206, the burr-ring 200 comprises a circumferentially positioned shoulder 208 that is arranged to cooperate with a guiding tube 600 according to the invention as will become apparent below (FIGS. 11-13). The shoulder can be of variable dimensions and is preferentially about 0.5 mm to 4.5 mm height and extends about 0.5 mm to 1 mm over the predetermined diameter of the cylindrical outer surface.

The body 202 comprises three cutaways gaps 216. As described above the cutaways gaps are arranged to be engaged by a tripod gripper in order to dismantle the burr-ring 200 from a chirurgical drill. Also the cutaways gaps 216 as shown in FIGS. 4-6 have the same flexibility features as described above for the cutaways gaps 116 of FIGS. 1-3.

The burr-ring 200 comprises clamping means in order to be mounted on a chirurgical drill 400. The clamping means are substantially the same as those described above and comprise at least one rib 212 arranged circumferentially of said central longitudinal hole 214 adapted. Each rib 212 is arranged to engage in a corresponding slot 404 arranged circumferentially on a chirurgical drill 400. The ribs 212 are formed by a circumferential diameter constriction of the central longitudinal hole 214 wherein the constriction extends on a predetermined length and preferentially extends about 1 mm. The constriction may scale about 0.5 mm with reference to the predetermined diameter of the central longitudinal hole 214. In a preferred embodiment, the constriction is arranged so that the diameter of the central longitudinal hole 214 is constricted to 2.20 mm.

Accordingly, a homologous slot 404, in which the ribs 212 can engage, has to be arranged circumferentially on the chirurgical drill 400. By engaging the slot 404, the ribs 212 clip onto the chirurgical drill 400. Once the ribs 212 are clipped into the slot 404, the burr-ring 200 and the chirurgical drill 400 are clamped together and are at most in a one-degree of freedom relationship that is a rotation around a common central longitudinal axis.

Both previously described embodiments of the burr-rings according to the invention are arranged to engage into a guiding tube that will be described in detail later. For now it should be noted that the guiding tube is arranged on a base frame of the type as described above and that it has an overall cylindrical shape. The guiding tube is comparable to a support structure that is the base for elements that engage in it. The burr-rings described herein have at least one-degree of freedom (translation) with respect to the guiding tubes and have preferably two-degrees of freedom (translation and rotation) with respect to the guiding tubes. The degrees of freedom described in this regard are relative to the guiding tube which is the base. In other words, the guiding tube is fixed and elements engaging in it may have degrees of freedom relative to said guiding tube. The degree of freedom relationship is so that the burr-ring constitutes the moving body (displacing element in motion) and the guiding tube constitutes the reference body (fixed base element).

FIG. 7 shows a burr-ring 300 according to another embodiment of the invention. Burr-ring 300 comprises a regular polygon body 302. The regular polygon body 302 lies on a circle of predetermined diameter preferentially comprised within a range going from 4 mm to 6.5 mm. In a preferred embodiment the predetermined diameter of the cylindrical outer surface of the cylindrical body 302 is of 5 mm 5 mm to 6.5 mm.

Analogous to what has been described above the burr-ring 300 comprises an upper side 306, a lower side 310, a central longitudinal hole 314, ribs 312 and cutaways gaps 316.

The burr-ring 300 does not comprise a shoulder as both previous described embodiments. The missing of a shoulder allows the chirurgical drill 400 in combination with the burr-ring 300 to penetrate deeply into an anatomical part. In fact, there is no abutment that is formed by the combination of a shoulder and a guiding tube. However, there is an abutment when the lower side 310 grounds onto the drill site.

It has to be noted that the shoulder may be missing in both previous described burr-rings 100 and 200 if one wants to penetrate deeply into the anatomical part by avoiding a mechanical abutment (a mechanical abutment is shown in FIG. 13: shoulder 108 lies onto guiding tube 604). However, security measures and sensitive controlling of the chirurgical procedure usually require the presence of said shoulders.

The burr-ring 300 of FIG. 7 may further comprise circumferential longitudinal ribs or circumferential longitudinal angles 318. When combining a burr-ring 300 comprising circumferential longitudinal angles 318 with a homologous regular-polygon shaped guiding tube there is a one-degree of freedom relationship between said guiding tube and said burr-ring 300 wherein said one-degree of freedom relationship is a longitudinal translation of the burr-ring 300. The homologous regular-polygon shaped guiding tube comprises circumferentially longitudinal ribs or angles that are arranged to center the burr-ring 300 in a predetermined position.

FIGS. 8-9 show a chirurgical drill 400 on which a burr-ring can be mounted. The chirurgical drill 400 has circumferentially arranged slot 404 in which clamping means (ribs) of a burr-ring can engage. Once the burr-ring 100, 200, 300 and the chirurgical drill 400 are mounted together there is a one-degree of freedom relationship between both elements wherein said one-degree of freedom relationship is a rotation around a common longitudinal central axis. The chirurgical drill 400 further comprises a drill head 406 and connecting means 408 to connect the drill 400 to an electric motor tool 2000 that provides the power for drilling (see FIG. 17).

FIG. 10 shows a chirurgical drill 500 on which a burr-ring can be mounted. The chirurgical drill 500 has circumferentially arranged slot 504 in which homologous clamping means (ribs) of a burr-ring can engage. Since the slot 504 has an increased surface (compared to slot 404), this embodiment is particularly adapted to mount fixedly a burr-ring onto a chirurgical drill; this can be done by glue bonding a burr-ring to the slot 504 for instance. In this case, there reigns a zero-degree of freedom relationship between the burr-ring and the chirurgical drill.

It has to be understood that one combination of two of the above described embodiments are incompatible. Specifically, this combination consists of a burr-ring fixedly mounted onto a chirurgical drill (zero-degree of freedom relationship between the burr-ring and the chirurgical drill) wherein at the same time the burr-ring and the guiding tube have a one-degree of freedom relationship that is only translational and not rotational (for example by using an angled burr-ring in combination with a homologous angled guiding tube). In fact, as a consequence this would implicate that there would be no rotation possibility for the chirurgical drill; there would be no rotational degree of freedom for the chirurgical drill and consequently no drilling would be possible. There has to be at least one rotational degree of freedom for the chirurgical drill in respect to the guiding tube. In other words, when there is a zero-degree of freedom relationship between the burr-ring and the chirurgical drill, there has to be a two-degree of freedom (rotation and translation) of the burr-ring in respect to the guiding tube in order to allow rotation of the drill.

FIG. 11 shows a schematic perspective view of a non assembled kit according to an embodiment of the present invention. The kit comprises a burr-ring 100, a chirurgical drill 400 and a base frame with a guiding tube 600.

It has to be noted that the base frame usually has an extended configuration that is homologous to an anatomical part of a patient. However, in the present embodiment the base frame is solely constituted by the guiding tube 600 and its features.

The guiding tube 600 has a cylindrical inner surface 602 of predetermined diameter. Preferentially, said predetermined diameter of the cylindrical inner surface is comprised between 5.1 mm and 7 mm (depending on the predetermined diameter of the burr-ring 100). The guiding tube further has a wall thickness and an outer surface 604.

The guiding tube 600 comprises a longitudinal cutout 606 extending along the entire guiding tube. The longitudinal cutout 606 provides direct view on the drilling site on one hand and allows a lateral access of the chirurgical drill 400 on the other hand. Preferentially, the longitudinal cutout 606 is arranged so that it forms a longitudinal opening along the guiding tube that is larger than the diameter of the drilling head 406.

The burr-ring 100 arranged to be placed around a chirurgical drill 400. The clamping means and the central longitudinal hole as described above provide first means for placement around a chirurgical drill 400; The slot 404 as described above provide second means for placement of the burr-ring 100 around the chirurgical drill 400. The burr-ring 100 further comprises a circumferential shoulder 108 on its upper side.

FIG. 12 shows a schematic perspective view of an assembled kit according to an embodiment of the present invention before a drilling procedure. The burr-ring 100 is positioned around the chirurgical drill 400 wherein both elements have a one-degree of freedom relationship to one another, and wherein said one-degree of freedom relationship is a rotation around a common longitudinal central axis. In other words, in this embodiment the burr-ring 100 and the chirurgical drill 400 are mounted in a rotational relationship to one another.

The burr-ring 100 has a cylindrical outer surface 102 of predetermined diameter of 5 mm. The diameter of the cylindrical outer surface 102 of the burr-ring is chosen so that it is slightly less than said diameter of said cylindrical inner surface 602 of the guiding tube 600 so that the burr-ring 100 can engage into the guiding tube 600. The cylindrical inner surface 602 may be of 5.1 mm. Further, the diameter of the burr-ring 100 is chosen such that the burr-ring 100 and the guiding tube 600 are at most in a two-degree of freedom relationship along the longitudinal axis of the guiding tube when the burr-ring 100 is engaged in the guiding tube 600. The degree of freedom relationship is so that the burr-ring 100 constitutes the moving body (displacing element in motion) and the guiding tube 600 constitutes the reference body (fixed base element).

FIG. 13 shows a schematic perspective view of an assembled kit according to an embodiment of the present invention during a drilling procedure (no jaw bone structure shown) and wherein the burr-ring 100 is engaged into the guiding tube 600. The circumferential shoulder 108 on the upper side of the burr-ring 100 form an abutment together with the upper side of said guiding tube 600 when said burr-ring 100 is fully engaged in the guiding tube 600.

FIG. 13 also shows direct view on the drilling site during a drilling procedure which is provided by the longitudinal cutout 606 of the guiding tube 600.

The frustoconical part 104 may assist the engagement of the burr-ring 100 into the guiding tube 600 and more precisely assist in the guidance of the burr-ring 100 (and consequently the drill 400) into an axial disposition.

Other embodiments can comprise a burr-ring without any circumferential shoulder. In these embodiments there is no abutment formed between the burr-ring and the guiding tube and the burr-ring can engage deeply into the guiding tube. However, eventually the lower side of the burr-ring will ground on the jaw bone structure.

The burr-ring and/or the guiding tube may comprise one or more measuring systems selected from the group consisting of: The metric system and The United States customary system or any other suitable measuring system. According to an embodiment, the burr-ring can comprise an inscription on its outer cylindrical surface and the outer cylindrical surface of the guiding tube can comprise a reference line.

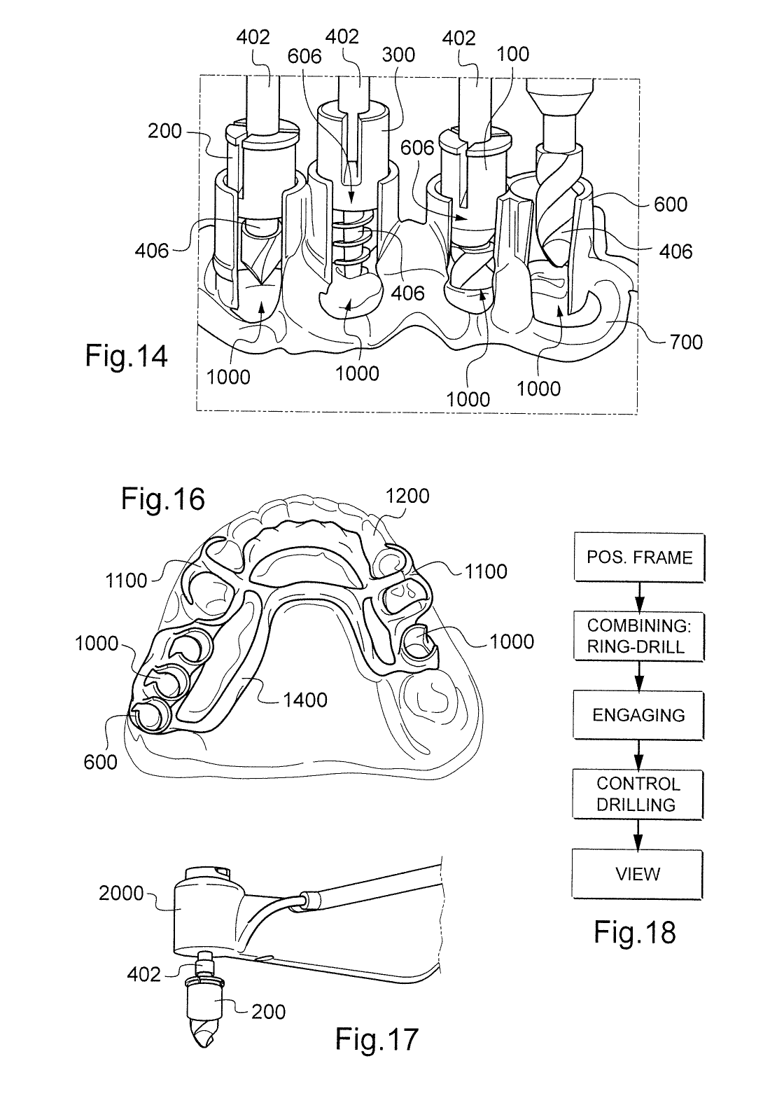

FIG. 14 shows a picture of a perspective view a kit according to an embodiment of the present invention. A base frame 700 is positioned on a synthetic reproduction of an anatomical part of a patient. The base frame 700 is homologous to said synthetic reproduction and consequently is homologous to a specific anatomical part of a patient. The base frame 700 comprises four adjacent guiding tubes 600 wherein each guiding tube comprises a longitudinal cutout 606 for direct view on the drilling site 1000. It has to be noted that there are as many drilling sites 1000 as there are guiding tubes 600.

FIG. 14 also shows three different burr-ring embodiments 100, 200 and 300 as respectively shown in FIGS. 1, 4 and 7.

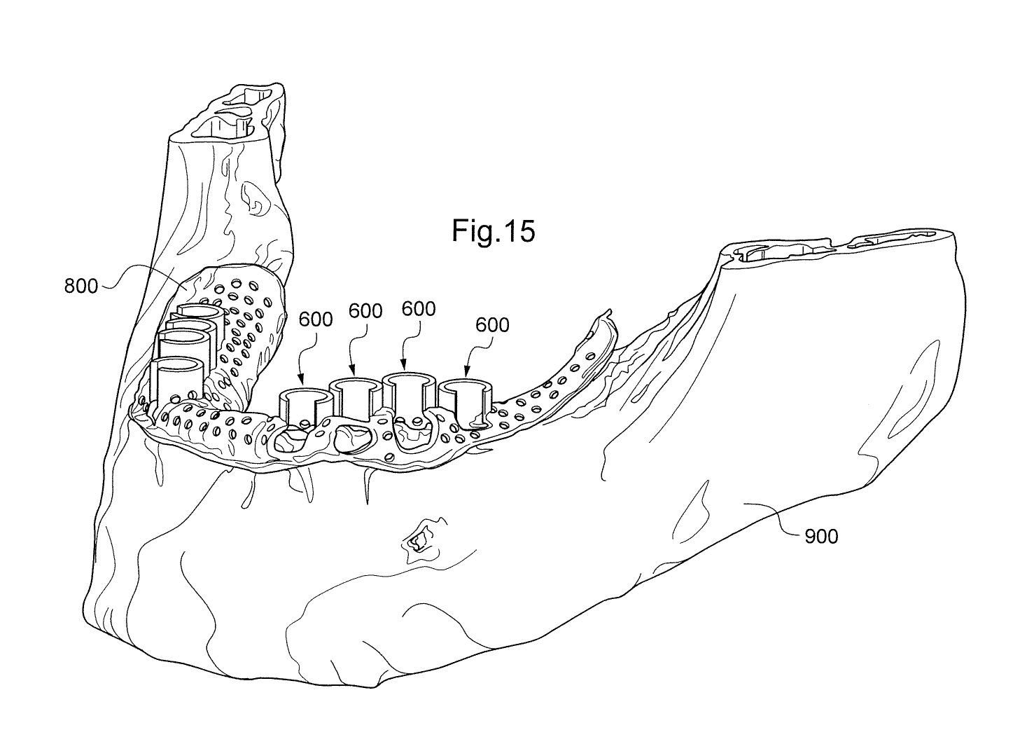

FIG. 15 shows a schematic picture of a perspective view of a base frame 800 according to another embodiment of the present invention placed on a lower jaw bone structure 900, wherein the base frame 800 comprises eight guiding tubes 600. The base frame 800 has a foot structure that is homologous to nearly the entire lower jaw bone structure 900 for providing a stable positioning during the chirurgical procedure.

FIG. 16 shows a picture of a top view of a base frame 700 according to another embodiment of the present invention placed on a synthetic jaw bone structure and dentition 1200. The base frame comprises a foot structure that is homologous to both the jaw bone structure and the dentition 1200. The base frame comprises positioning means 1100 that surround at least partially selected teeth. The base frame further comprises stabilizing means 1400 that support the overall structure of the base frame and provide stability during the chirurgical procedure.

It has to be noted that FIGS. 14, 15 and 16 make reference to a lower jaw bone structure. Same features apply to other embodiments where the kit is used with chirurgical procedures in which implants are to be placed on an upper jaw bone structure.

FIG. 17 shows a picture of a perspective view of a burr-ring 200 placed around a chirurgical drill 400 combined with an electric motor tool 2000.

FIG. 18 shows a flowchart of a method for bone surgery according to the invention. A first step POS. FRAME comprises placing a base frame 700 having at least one guiding tube 600 on an anatomical part of a patient. The frame, as it is described above provides the positioning of a chirurgical drill 400 on said anatomical part. The guiding tube has a cylindrical inner surface 602 of predetermined diameter and a longitudinal cutout 606. The guiding tube is of cylindrical shape and consequently has a central longitudinal axis.

The next step COMBINING: RING-DRILL comprises the placement of burr-ring 100 around said chirurgical drill 400. The burr-ring 100 comprises a cylindrical outer surface 102 of predetermined diameter that is slightly less than the diameter of the cylindrical inner surface 602 of the guiding tube 600. Once the burr-ring 100 is placed around the chirurgical drill 400, both have a common central longitudinal axis. As it is described above, the burr-ring and the drill may have a one- or zero-degree of freedom relationship to one another.

The next step ENGAGING comprises the engagement of the chirurgical drill 400 and the burr-ring 100 into the guiding tube 600 (see FIG. 12). The engagement can be axially (from above) or laterally (via the longitudinal cutout 606 of the guiding tube 600).

The next step CONTROL DRILLING comprises an axial penetration of the chirurgical drill 400 into the anatomical part of a patient (see FIG. 13). The penetration is controlled along a central longitudinal axis that is common to the chirurgical drill 400, the burr-ring 100 and to the guiding tube 600. The drilling is mainly controlled by the burr-ring engaging into said guiding tube. As described above, the burr-ring 100 and the guiding tube 600 are at most in a two-degree of freedom relationship and wherein said two-degree of freedom relationship is consisting of a rotation and a translation of the burr-ring along the common central longitudinal axis. This step CONTROL DRILLING goes along with another step VIEW that comprises direct viewing of the drilling site. The viewing is assured by longitudinal cutout 606 of the guiding tube 600.

As described above, it has to be noted that a zero-degree of freedom relationship between a burr-ring and a chirurgical drill is not compatible with a one-degree of freedom relationship between the burr-ring and the guiding tube wherein said one-degree of freedom relationship is a translation along the common longitudinal axis. The needs to be at least degree of freedom that is a rotation around said common longitudinal axis in order to allow drilling.

Also it has to be noted that that the invention is described herein with reference to a chirurgical drill. Naturally, the terminology “chirurgical drill” has to be understood in a larger sense that includes various chirurgical instruments such as an osteotome for instance or any other kind of instrument used in implantology or osteoplastic procedures. As a consequence, “chirurgical drills” that fall within the scope of protection of the present invention are chirurgical instruments that are used in combination with a burr-ring and a guiding tube as described above.

1. Kit for bone surgery comprising a chirurgical drill and a base frame for positioning of said chirurgical drill on an anatomical part of a patient,
wherein said base frame comprises at least one guiding tube with a cylindrical inner surface having a predetermined diameter and a longitudinal cutout, the kit further comprising a burr-ring arranged to be placed around said chirurgical drill, said burr-ring comprising a cylindrical outer surface having a predetermined diameter,
wherein the diameter of said cylindrical outer surface of the burr-ring is slightly less than said diameter of said cylindrical inner surface of the guiding tube and such that the burr-ring and the guiding tube are at most in a two-degree of freedom relationship along the longitudinal axis of the guiding tube when the burr-ring is engaged in the guiding tube, while the longitudinal cutout provides for direct view of the drilling site.

2. Kit according to claim 1 wherein said burr-ring comprises a central longitudinal hole of predetermined diameter substantially complementary to an outer diameter of said chirurgical drill so that said burr-ring can be placed around said chirurgical drill.

3. Kit according to claim 2 wherein said burr-ring and said chirurgical drill are in a one-degree of freedom relationship when the burr-ring is placed around the chirurgical drill and wherein said one-degree of freedom relationship is a rotation around a common longitudinal axis.

4. Kit according to claim 2 wherein said burr-ring and said chirurgical drill are mounted fixedly together in a zero-degree of freedom relationship when the burr-ring is placed around the chirurgical drill.

5. Kit according to claim 1 wherein the burr-ring comprises clamping means comprising at least one rib arranged circumferentially of said central longitudinal hole adapted to engage in at least one slot arranged circumferentially on said chirurgical drill.

6. Kit according to claim 1 wherein said burr-ring comprises an at least partially circumferential shoulder on an upper side to form an abutment together with the upper side of said guiding tube when the burr-ring is introduced in the guiding tube.

7. Kit according to claim 1 wherein said two-degree of freedom relationship of said burr-ring and said guiding tube is set along a common longitudinal axis and consisting of a longitudinal translation and a rotation.

8. Kit according to claim 1 wherein the burr-ring comprises at least one circumferential longitudinal rib on its cylindrical outer surface and the guiding tube comprises at least one circumferential longitudinal slot on its cylindrical inner surface so that the burr-ring and the guiding tube are in a one-degree of freedom relationship consisting of a longitudinal translation when said burr-ring is engaged in said guiding tube.

9. Kit according to claim 1 wherein the burr-ring comprises at least one circumferential longitudinal slot on its cylindrical outer surface and the guiding tube comprises at least one circumferential longitudinal rib on its cylindrical inner surface so that the burr-ging and the guiding tube are in a one-degree of freedom relationship consisting of a longitudinal translation when said burr-ring is engaged in said guiding tube.

10. Kit according to claim 1 wherein the longitudinal cutout extends along the entire guiding tube.

11. Method for bone surgery comprising the steps of:
a. placing a base frame comprising at least one guiding tube with a cylindrical inner surface having a predetermined diameter and a longitudinal cutout on an anatomical part of a patient for providing positioning of a chirurgical drill on said anatomical part,
b. placing a burr-ring around said chirurgical drill wherein said burr-ring comprises a cylindrical outer surface having a predetermined diameter that is slightly less than said diameter of said cylindrical inner surface of said guiding tube,
c. drilling into said anatomical part of the patient wherein the chirurgical drill penetrates axially into the anatomical part along a common central longitudinal axis while said burr-ring engages into said guiding tube and wherein the burr-ring and the guiding tube are at most in a two-degree of freedom relationship and wherein said two-degree of freedom relationship is consisting of a rotation and a translation of the burr-ring along said common central longitudinal axis, and
d. controlling the drilling by providing direct view of the drilling site by said longitudinal cutout of the guiding tube.

12. Burr-ring comprising a body of generally cylindrical shape having a central longitudinal hole of predetermined diameter wherein said central longitudinal hole comprises at least one rib consisting of a circumferential diameter constriction of predetermined length.

13. Burr-ring according to claim 12 wherein said body has a cylindrical outer surface and comprises a shoulder that is at least partially arranged circumferential on said cylindrical outer surface.

14. Burr-ring according to claim 12 wherein said body comprises a terminal frustoconical part of a decreasing diameter and wherein said decreasing diameter decreases from substantially the diameter of said body to substantially the predetermined diameter of said hole.

15. Base frame comprising a foot structure homologous to an anatomical part of a patient and at least one guiding tube with a cylindrical inner surface of predetermined diameter, said guiding tube comprising a longitudinal cutout.

16. Base frame according to claim 15 wherein the longitudinal cutout extends along the entire guiding tube.

17. Base frame according to claim 15 wherein said foot structure is homologous to an anatomical part chosen in the group consisting of an upper jaw bone structure, a lower jaw bone structure or at least a part of a dentition.

18. Burr-ring according to claim 13 wherein said body comprises a terminal frustoconical part of a decreasing diameter and wherein said decreasing diameter decreases from substantially the diameter of said body to substantially the predetermined diameter of said hole.
